(19) United States
(12) Patent Application Publication
(10) Pub. No.: US 2025/0268836 A1
(43) Pub. Date: Aug. 28, 2025

(54) MICROSPHERE FORMULATIONS COMPRISING MULTIPLE NON-IDENTICAL PEPTIDES AND METHODS FOR MAKING THE SAME

(71) Applicant: Flow Pharma, Inc., Warrensville Heights, OH (US)

(72) Inventors: Bradley Minrovic, Willoughby, OH (US); Colin Spencer, Oakwood Village, OH (US); Nicholas Delucia, Oakwood Village, OH (US); Scott R. Burkholz, Oakwood Village, OH (US); Reid Rubsamen, Oakwood Village, OH (US)

(73) Assignee: Flow Pharma, Inc., Warrensville Heights, OH (US)

(21) Appl. No.: 18/550,416

(22) PCT Filed: Apr. 22, 2022
(86) PCT No.: PCT/US22/71868

§ 371 Date: Sep. 13, 2023

Related U.S. Application Data
(60) Provisional application No. 63/178,888, filed on Apr. 23, 2021.

(51) Int. Cl. A61K 9/50; A61K 9/10; A61K 38/08; A61K 39/00

(52) U.S. Cl. CPC A61K 9/5031; A61K 9/10; A61K 9/5089; A61K 38/08; A61K 39/0005; A61K 2039/55561

(57) ABSTRACT

A microsphere formulation comprising polymer microspheres is provided, each polymer microsphere comprising: at least two non-identical peptides; and a biodegradable polymer, wherein each polymer microsphere has a drug load of at least about 0.15 wt/wt % of each of the non-identical peptides, and wherein the polymer microspheres have an average particle size of less than about 12.6 μM (D₅₀). The polymer microspheres may further comprise an adjuvant. Methods for making and using the microsphere formulations are also provided.

### CROSS-REFERENCE TO RELATED APPLICATIONS

This application claims priority from U.S. Provisional Patent Application No. 63/178,888, filed on Apr. 23, 2021, which is incorporated by reference herein in its entirety.

### SEQUENCE LISTING

A Sequence Listing has been submitted electronically in ASCII format and is hereby incorporated by reference in its entirety. The ASCII copy, created on Nov. 28, 2023, is named 52413-00029 SQT.txt and is 842 bytes in size.

### BACKGROUND

Microspheres containing peptides that are capable of eliciting an immune response (that is, capable of acting as an antigen) are known to be effective as drug delivery systems. For example, by loading a sufficient amount of a peptide into a microsphere having a specific size range and introducing the microsphere into a subject, the subject's T-cells may engulf the microsphere. The subject's T-cells may break down the whole microsphere, resulting in the peptide antigen being released.

To date, only one peptide type is loaded into each microsphere. If non-identical peptides are desired to be introduced to the subject, then a different peptide type is loaded into each microsphere. Thus, for example, if peptide A does not provide the desired response inside the first microsphere to enter the T-cell, the T-cell would “clear” the microsphere and peptide A. A second microsphere containing peptide B would follow. If peptide B does not provide the desired response inside the second microsphere to enter the T-cell, the T-cell would “clear” the microsphere and peptide B. This process continues for peptides C, D, and so on.

Until now, loading multiple non-identical peptide types (e.g., A, B, and C) into a single microsphere has been avoided, at least in part to avoid competitive binding between the peptide types.

### SUMMARY

A microsphere formulation is provided, the microsphere formulation comprising at least two non-identical peptides.

In one aspect, the microsphere formulation comprises polymer microspheres, each polymer microsphere comprising: at least two non-identical peptides; and a biodegradable polymer, wherein each polymer microsphere has a drug load of at least about 0.15 wt/wt % of each of the non-identical peptides, and wherein the polymer microspheres have an average particle size of less than about 12.6 μM (D₅₀). In one aspect, the polymer microspheres further comprise an adjuvant.

In one aspect, the microsphere formulation may be prepared by a method, the method comprising: (A) mixing: (i) a peptide solution comprising at least two non-identical peptides; (ii) an adjuvant solution comprising an adjuvant; and (iii) a polymer solution comprising a biodegradable polymer, to form a dispersed phase; (B) mixing: (i) water; and (ii) a surfactant, to form a continuous phase; and (C) combining the dispersed phase with the continuous phase in a homogenizer.

### BRIEF DESCRIPTION OF THE FIGURES

In the accompanying figures, structures are illustrated that, together with the detailed description provided below, describe example aspects of the claimed invention. Elements shown as a single component may be replaced with multiple components, and elements shown as multiple components may be replaced with a single component. The figures are not necessarily to scale, and the proportion of certain elements may be exaggerated for the purpose of illustration.

FIG. 1 is an example schematic representation of a method for making microsphere formulations comprising polymer microspheres comprising at least two non-identical peptides.

FIG. 2 is an example scanning electron microscopy (SEM) image of polymer microspheres made according to the method represented in FIG. 1.

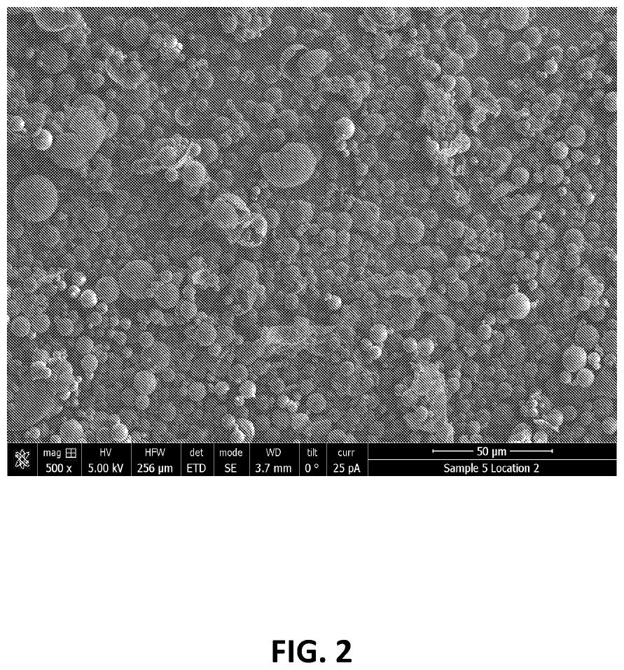

### DETAILED DESCRIPTION

Microsphere formulations are provided, the microsphere formulations comprising multiple non-identical peptides that may be useful for eliciting an immune response. Methods for making the microsphere formulations are also provided.

In one aspect, the microsphere formulation comprises polymer microspheres, each polymer microsphere comprising: at least two non-identical peptides; and a biodegradable polymer, wherein each polymer microsphere has a drug load of at least about 0.15 wt/wt % of each of the non-identical peptides, and wherein the polymer microspheres have an average particle size of less than about 12.6 μM (D₅₀). In one aspect, the polymer microspheres further comprise an adjuvant.

In one aspect, the microsphere formulation may be prepared by a method, the method comprising: (A) mixing: (i) a peptide solution comprising at least two non-identical peptides; (ii) an adjuvant solution comprising an adjuvant; and (iii) a polymer solution comprising a biodegradable polymer, to form a dispersed phase; (B) mixing: (i) water; and (ii) a surfactant, to form a continuous phase; and (C) combining the dispersed phase with the continuous phase in a homogenizer.

### Peptides

In some aspects, the microsphere formulations may include three non-identical peptides. In one aspect, the peptides are chosen for their ability to elicit an immune response to COVID-19. Examples of the peptides include, but are not limited to, T-lymphocyte surface antigen acetate salts SEQ ID NO: 1 (Formula: C₆₇H₈₃N₁₃O₁₄; MW: 1,294.49), SEQ ID NO: 2 (Formula: C₄₉H₈₈N₁₄O₁₄; MW: 1,097.33), and SEQ ID NO: 3 (Formula: C₄₉H₇₇N₁₁O₁₄, MW: 1,044.23):

SEQ ID NO: 1:
L-S-P-R-W-Y-F-Y-Y
SEQ ID NO: 2:
L-L-L-D-R-L-N-Q-L
SEQ ID NO: 3:
K-T-F-P-P-T-E-P-K.

Of course, the method may be used to formulate vaccines for other illnesses, using peptides specifically chosen for each illness. The peptides disclosed herein are for example purposes only and are not meant to limit the disclosure.

### Adjuvants

The peptides may be combined with an adjuvant, such as CpG 1018, a toll-like receptor 9 (TLR9) agonist adjuvant commercially available from Dynavax, D-(+)-Mannose, or D-mannitol (or another suitable sugar component).

### Biodegradable Polymers

Suitable biodegradable polymers may include a polylactic acid (a “PLA”), a poly(lactic-co-glycolic acid) (a “PLGA”), a polyesteramide, a polyanhydride, a polyacetal, a poly(ortho ester), a polyphosphoester, a polycaprolactone, and a polycarbonate. In some aspects, the biodegradable polymer comprises a PLGA. In some aspects, the biodegradable polymer may comprise a copolymer having a co-monomer ratio for lactide to glycolide content of about 50:50 to about 85:15. In other aspects, the biodegradable polymer comprises a PLA.

The polymer may be acid end-capped or ester end-capped. The biodegradable polymer may have an average molecular weight of from about 30 kDa to about 300 kDa, or in another aspect, about 31 kDa to about 278 kDa. In one aspect, the inherent viscosity (IV) of the polymer may be between about 0.1 to about 1.8 dl/g. In one aspect, the IV of the polymer may be between about 0.1 and about 0.3 dl/g.

In one aspect, the biodegradable polymer is a PLGA. In one aspect, the PLGA comprises Viatel™ DLG 7502A, acid terminated, IV=0.20 dL/g, supplied by Ashland (“DLG 7502A”).

### Dispersed Phase

In one aspect, the peptides, adjuvant composition, and the biodegradable polymer may be dissolved separately in suitable solvents or co-solvent mixtures and combined to form a dispersed phase. The formation of the dispersed phase may be accomplished using various solvent systems, with solvents necessary to solubilize the peptides, the adjuvant composition, and the biodegradable polymer. Suitable solvents may include, for example, methylene chloride (also known as dichloromethane or DCM), dimethyl sulfoxide (DMSO), methanol, ethyl acetate, acetic acid, acetone, acetonitrile, acetyl acetone, acrolein, acrylonitrile, allyl alcohol, 1,3-butanediol, 1,4-butanediol, 1-butanol, 2-butanol, tert-butanol, 2-butoxyethanol, n-butyl amine, butyl dioxitol acetate, butyraldehyde, butyric acid, 2-chloroethanol, diacetone alcohol, diacetyl, diethylamine, diethylene glycol diethyl ether, diethylene glycol dimethyl ether, diethylene glycol monobutyl ether, diethylene glycol monobutyl ether acetate, diethylene glycol monoethyl ether, diethylene glycol monoethyl ether acetate, diethylene glycol monomethyl ether, N,N-diethylnicotinamide, N,N-dimethylacetamide, N,N-dimethylformamide, 1,4-dioxane, 2-ethoxyethanol, 2-ethoxyethyl acetate, ethyl formate, ethylene glycol methyl ether acetate, formic acid, furfural, glycofurol, hexylene glycol, isobutanol, isopropyl alcohol, 2,6-lutidine, methyl acetate, methyl ethyl ketone, methyl isopropyl ketone, methyl propionate, N-methylpyrrolidone, morpholine, tert-pentanol, 2-picoline, 3-picoline, 4-picoline, piperidine, 1-propanol, propionaldehyde, propylene oxide, pyridine, pyrimidine, pyrrolidine, tetrahydrofuran, tetramethylurea, triacetin, triethylene glycol, and trimethyl phosphate, and combinations thereof.

In some aspects, the peptides are dissolved in DMSO, the adjuvant composition is dissolved in methanol, and the polymer composition is dissolved in DCM. In those examples where the peptides, the adjuvant composition, and the polymer composition are first separately dissolved, once dissolved, the components may be combined. In one aspect, the polymer is dissolved in DCM and a sugar, if included, is dissolved in DMSO. The polymer and the sugar may then be combined with the peptide composition and the adjuvant composition. In one aspect, the ratio of DCM:DMSO:methanol is about 9:1:1. In one aspect, the ratio of DCM:DMSO:methanol is about 9:2:1.

The organic solvent is removed from the microspheres in the course of their preparation. A microsphere is considered to be “essentially free” of organic solvent if the microsphere meets the standards set forth in the “ICH Harmonised Guideline, Impurities: Guideline for Residual Solvents Q3C(R8), Current Step 4 version dated 22 Apr. 2021,” which is incorporated herein by reference in its entirety.

### Continuous Phase

The dispersed phase may be combined with an aqueous continuous phase that comprises water and, optionally, a surfactant. The surfactant component may be present in the continuous phase in an amount of about 0.35% to about 1.0% by weight in water. In one aspect, the surfactant component comprises polyvinyl alcohol (“PVA”) in a concentration of about 0.35% by weight in water.

In some aspects, the dispersed phase flow rate to the homogenizer may be from about 10 mL/min to about 120 mL/min, including about 100 mL/min. In some aspects, the continuous phase flow rate to the homogenizer may be from about 2 L/min to about 8 L/min. Thus, in one aspect, the continuous phase:dispersed phase ratio may be from about 66:1 to about 200:1, including about 100:1 and about 80:1.

The continuous phase may be provided at room temperature or above or below room temperature. In some aspects, the continuous phase may be provided at about 40° C., about 37° C., about 35° C., about 30° C., about 25° C., about 20° C., about 15° C., about 10° C., about 5° C., about 0° C., and any range or value between any of those temperature values.

### Homogenizer

For brevity, and because the methods are equally applicable to either, the phrase “homogenizer” contemplates a system or apparatus that can homogenize the dispersed phase and the continuous phase, emulsify the dispersed phase and the continuous phase, or both, which systems and apparatuses are known in the art. For example, in one aspect, the homogenizer is an in-line Silverson Homogenizer (commercially available from Silverson Machines, Waterside, UK) or a Levitronix® BPS-i100 integrated pump system used, e.g., as described in U.S. Pat. No. 11,167,256, which is incorporated by reference herein in its entirety. In one aspect, the homogenizer is a membrane emulsifier. In one aspect, the homogenizer runs at an impeller speed of about 1,000 to about 10,000 revolutions per minute (“RPM”), including about 2,000 RPM, about 3,000 RPM, about 4,000 RPM, about 5,000 RPM, about 6,000 RPM, about 7,000 RPM, about 8,000, about 9,000 RPM, about 10,000 RPM, or any value or range between any of those RPM values.

### Drug Load

In one aspect, the drug load of each polymer microsphere in a drug to polymer ratio, expressed as a percentage, of at least about 0.15 wt/wt % up to about 1.0 wt/wt % of each of the non-identical peptides.

In another aspect, the drug load of each polymer microsphere in a drug to polymer ratio, expressed as a percentage, may range from about 0.1 wt/wt % to about 1.0 wt/wt % for each of the peptides, about 0.01 wt/wt % to about 0.2 wt/wt % for the adjuvant composition, and up to about 1.0 wt/wt % for the sugar component.

### Particle Size

The polymer microspheres may be any size that is safely and efficaciously injectable. In one aspect, the polymer microspheres may have an average particle size of about 5 μm to about 12.6 μm (D₅₀), about 7 μm (D₅₀), and less than about 12.6 μm (D₅₀).

### EXAMPLES

### Example 1

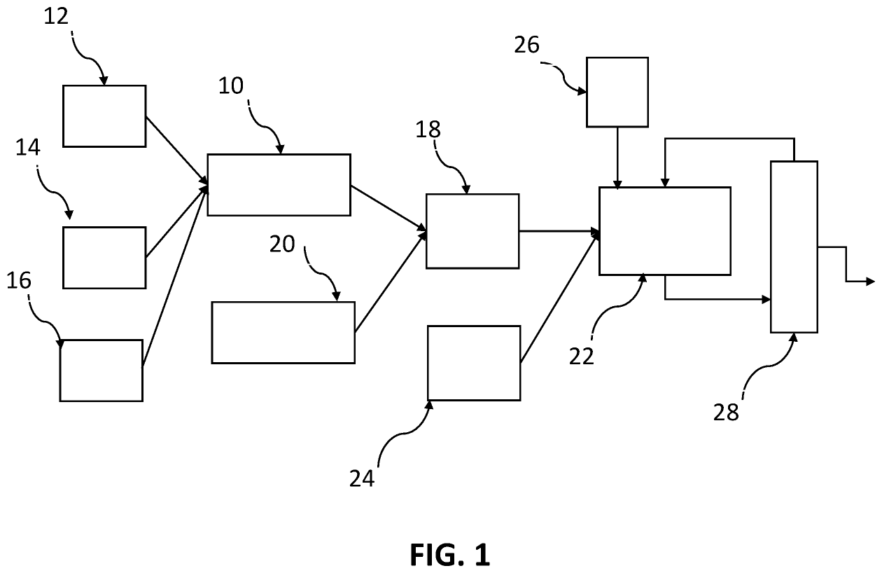

With reference to FIG. 1, the microsphere formulation may be prepared using a continuous water-in-oil (W/O) emulsification/solvent extraction procedure. In one example, one 125 g batch was made according to the following procedure.

The dispersed phase 10 comprises a combination of a peptide solution 12, an adjuvant solution 14, and a polymer solution 16. The peptide solution 12 may be prepared by combining 1.25 g each of LY9, LL9, and KK9 peptides (each nine amino acids in length) in a glass vessel. To that vessel, 250 g of DMSO is added. The peptide solution 12 is stirred until the peptides are dissolved.

The adjuvant solution 14 may include 125 mg of CpG 1018 (an oligonucleotide), which is combined with 250 g of methanol. The adjuvant solution is stirred until the adjuvant is dissolved.

The polymer solution 16 may be formed by combining 119.875 g of DLG 7502A from Ashland Chemicals with a monomer ratio of 75:25 and an inherent viscosity of about 0.20 dL/g, with 2250 g of dichloromethane and 1.25 g of D-(+)-mannose.

Each component of the dispersed phase 10 may be filtered using a 0.2 micron sterilizing PTFE or PVDF membrane filter (such as EMFLON, commercially available from Pall or SartoriousAG).

The peptide solution 12, the adjuvant solution 14, and the polymer solution 16 are combined in a holding vessel to form the dispersed phase 10. The dispersed phase 10 is pumped into a homogenizer 18, such as an in-line Silverson Homogenizer (commercially available from Silverson Machines, Waterside UK) or a Levitronix Homogenizer (as described in US2021/0001290A1, which is incorporated by reference herein in its entirety), at a defined flow rate. In one aspect, the defined flow rate for the dispersed phase is 100 mL/min.

Simultaneously, a continuous phase 20 including an aqueous solution containing a surfactant, such as PVA, is also pumped into the homogenizer 18 at a defined flow rate. In one aspect, the flow rate for the continuous phase 20 is about 8.0 L/min. The speed of the homogenizer 18 is generally fixed to achieve a desired microsphere size distribution. A suitable continuous microsphere process is described in U.S. Pat. No. 5,945,126, which is incorporated by reference herein in its entirety. In one aspect, the homogenizer 18 is configured to operate at a speed of 6000 rpm.

The formed or forming microspheres exit the homogenizer and enter a solvent removal vessel (SRV) 22. Water may be added to SRV 22 during microsphere formation from a water dilution composition vessel 24 in order to minimize the organic solvent level in the aqueous medium. The resulting suspension is mixed in the SRV 22 during the microsphere formation period. After the dispersed phase 10 has been exhausted, the continuous phase 20 and water dilution composition 24 pumps are stopped, and the washing steps are initiated. Solvent removal may be achieved using water washing 26 and a hollow fiber filter (commercially available as HFF from GE Healthcare) 28. An example system is described in U.S. Pat. No. 6,270,802, which is incorporated by reference herein in its entirety.

The washing steps begin by washing the microsphere suspension with room temperature water for about 50 minutes. The washed microspheres are collected and freeze-dried overnight in a lyophilizer (Virtis) to remove moisture. The resulting microspheres are a freeflowing off-white bulk powder.

In use, the microspheres may be suspended in a diluent for administration. The diluent may generally contain a thickening agent, a tonicity agent, and a surfactant. The thickening agent may include carboxymethyl cellulose-sodium (CMC-Na) or other suitable compounds. An appropriate viscosity grade and suitable concentration of CMC-Na may be selected so that the viscosity of the diluent is 3 cps or higher. Generally, a viscosity of about 10 cps is suitable, however a higher viscosity diluent may be preferred for larger microspheres in order to minimize the settling of microspheres in the suspension.

Uniform microsphere suspensions without particle settling will result in a consistent delivered dose during drug administration by injection or by nebulizer in a mist form. To achieve a tonicity of the diluent closer to the biological system, i.e., about 290 milliosmole (mOsm), solutes such as mannitol, sodium chloride, or any other acceptable salt may be used. The diluent may also contain a buffer salt to maintain the pH of the composition. Typically, the pH is maintained around neutral by adjusting the buffer content as needed (pH 7 to about 8). In one aspect, the diluent comprises about 134 g of CMC-Ns, about 6.6 g polysorbate 20, and about 660 g of mannitol in a 10 kg aqueous solution. Example resulting particle size and drug loads are provided below in Table 1 and Table 2.

TABLE 1

| Polymer | Polymer | | Ashland DLG 7502 A | | |
|---|---|---|---|---|---|
| | Monomer Ratio | | 75:25 | | |
| | Polymer IV (dL/g) | | 0.20 | | |
| Formulations | Batch Size (g) | | 125 | | |
| | DCM Weight (g) | | 2,250 | | |
| | DMSO Weight (g) | | 250 | | |
| | MeOH Weight (g) | | 250 | | |
| | Homogenizer Mixing Speed | | 6000 | | |
| | (RPM) | | | | |
| Analytical | Residual Solvent | | ND/ND/ND* | | |
| | DCM/DMSO/MeOH (%) | | | | |
| | Particle | Tray | 1 | 2 | 3 |
| | Size (μm) | Dᵥ10 | 3.5 | 3.7 | 3.5 |
| | | Dᵥ50 | 6.7 | 7.0 | 6.9 |
| | | Dᵥ90 | 12.6 | 13.3 | 14.0 |
| | Moisture Content (%) | | 5.1 | | |
| | Molecular Weight (kDa) | | 21.8 | 21.9 | 21.6 |

*“ND” means “not detected”

TABLE 2

| API | CpG | KK9 | LL9 | LY9 |
|---|---|---|---|---|
| Target Drug | 0.1 | 1.0 | 1.0 | 1.0 |
| Load (%) | | | | |
| Actual Drug | 0.06 | 0.53 | 0.60 | 0.98 |
| Load (%) | | | | |

### Example 2

In a second example, a 5 g batch was prepared generally using the method described above and according to Table 3 and Table 4 below.

TABLE 3

| Polymer | Polymer | | Ashland 7502 A |
|---|---|---|---|
| | Monomer Ratio | | 75:25 |
| | Polymer IV (dL/g) | | 0.20 |
| Formulations | Batch Size (g) | | 5 |
| | DCM Weight (g) | | 90 |
| | DMSO Weight (g) | | 10 |
| | MeOH Weight (g) | | 10 |
| | Homogenizer Mixing Speed (RPM) | | 6000 |
| Analytical | Particle Size (μm) | Dᵥ10 | 2.1 |
| | | Dᵥ50 | 7.3 |
| | | Dᵥ90 | 16.8 |

TABLE 4

| API | CpG | KK9 | LL9 | LY9 | Mannitol |
|---|---|---|---|---|---|
| Target Drug | 0.11 | 1.0 | 1.0 | 0.7 | 0.05 |
| Load (%) | | | | | |
| Actual Drug | 0.07 | 0.69 | 0.74 | 0.62 | ND |
| Load (%) | | | | | |

### Example 3

In a third example, a 125 g batch was prepared generally using the method described above and according to Tables 5, 6, and 7 below.

TABLE 5
Formulation Parameters

| Component | Value |
|---|---|
| Batch Size (g) | 125 |
| Polymer co-monomer ratio | 75:25 |
| Polymer Type | Ashland 7502 A |
| Polymer IV (dL/g) | 0.20 |
| DCM Amount (g) | 2250 |
| DMSO Amount (g) | 500 |
| MeOH Amount (g) | 250 |
| CP PVA % | 0.35 |
| Homogenizer Speed (RPM) | 6000 |
| CP Flow Rate (L/min) | 8 |
| DP Flow Rate (mL/min) | 100 |
| Water Flow Rate (L/min) | 8 |
| LL9 Target Drug Load (%) | 1.0 |
| KK9 Target Drug Load (%) | 1.0 |
| LY9 Target Drug Load (%) | 1.0 |
| Mannitol Target Drug Load (%) | N/A |
| CpG Target Drug Load (%) | 0.1 |

TABLE 6
Processing Parameters

| Component | Value |
|---|---|
| Batch Size (g) | 125 |
| Washing Flow Rate (L/min) | 6 |
| Washing Suspension Volume (L) | 30 |
| Washing Temperature (° C.) | 25 |
| Washing Time (min) | 50 |
| Wet Sieve Pore Size (μm) | 250 |
| Mannitol Concentration in Diluent (wt. %) | 10.0 |
| Na-CMC Concentration in Diluent (wt. %) | 2.0 |
| Polysorbate-80 Concentration in Diluent | 0.10 |
| (wt. %) | |
| IP Sample Mass Concentration (mg/g) | 50.985 |
| Target Mass Concentration (mg/g) | 16.667 |

TABLE 1
Analytical Characteristics

| Component | Value | | | |
|---|---|---|---|---|
| Total Batch Yield (%) | 60.5 | | | |
| LL9 Drug Load (%) | 0.66 | | | |
| KK9 Drug Load (%) | 0.54 | | | |
| LY9 Drug Load (%) | 0.61 | | | |
| Mannitol Drug Load (%) | N/A | | | |
| CpG Drug Load (%) | 0.02 | | | |
| Particle Sizing | DIA-Vol % | LD-1 | LD-2 | DIA-Num % |
| D₁₀ | 4.4 | 2.6 | 2.7 | 1.2 |
| D₅₀ | 8.5 | 6.7 | 7.0 | 3.1 |
| D₉₀ | 14.4 | 16.6 | 17.8 | 7.3 |

DIA-Vol % means dynamic image analysis, volume distribution. DIA-Num % means dynamic image analysis number distribution. LD-1 and LD-2 are laser diffraction tests conducted on laser diffraction apparatuses.

By using the method disclosed herein, the microsphere formulation will have target drug loads of about 0.7% to about 1.0% and actual drug loads of at least about 0.5% to about 0.98% for each of the peptides. Peptides generally do not have a long half-life once the protective microspheres are broken down inside of the T-cells. It is therefore desirable to increase the drug load of each peptide so that they remain inside the T-cell long enough for the competitive nature of the inhabitation process to identify the peptide most likely to produce the desired immune response.

While the present application has been illustrated by the description of specific aspects thereof, e.g., with respect to an example including peptides chosen for their ability to elicit an immune response to COVID-19, the applicant does not intend to so restrict or in any way limit the scope of the appended claims. Indeed, the invention may be used to enable any aspect or embodiment disclosed in U.S. Provisional Patent Application No. 63/178,912, which is incorporated by reference herein in its entirety. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

Unless otherwise specified, “a,” “an,” “the,” “one or more of,” and “at least one” are used interchangeably. The singular forms “a”, “an,” and “the” are inclusive of their plural forms. The recitations of numerical ranges by endpoints include all numbers subsumed within that range (e.g., 1 to 5 includes 1, 1.5, 2, 2.75, 3, 3.80, 4, 5, etc.). The terms “comprising” and “including” are intended to be equivalent and open-ended. The phrase “consisting essentially of” means that the composition or method may include additional ingredients and/or steps, but only if the additional ingredients and/or steps do not materially alter the basic and novel characteristics of the claimed composition or method. The phrase “selected from the group consisting of” is meant to include mixtures of the listed group.

When reference is made to the term “each,” it is not meant to mean “each and every, without exception.” For example, if reference is made to microsphere formulation comprising polymer microspheres, and “each polymer microsphere” is said to have a particular peptide content, if there are 10 polymer microspheres, and only one or two of the polymer microspheres have the particular peptide content, then that subset of one or two polymer microspheres is intended to meet the limitation.

The term “about” in conjunction with a number is intended to include ±10% of the number. This is true whether “about” is modifying a stand-alone number or modifying a number at either or both ends of a range of numbers. In other words, “about 10” means from 9 to 11. Likewise, “about 10 to about 20” contemplates 9 to 22 and 11 to 18. In the absence of the term “about,” the exact number is intended. In other words, “10” means 10.

What is claimed is:

1. A microsphere formulation, comprising:
polymer microspheres, each polymer microsphere comprising:
a. at least two non-identical peptides; and
b. a biodegradable polymer,
wherein each polymer microsphere has a drug load of at least about 0.1 wt/wt % of each of the non-identical peptides, and
wherein the polymer microspheres have an average particle size (D₅₀) of less than about 12.6 μm.

2. The microsphere formulation of claim 1, wherein each polymer microsphere has a drug load of between about 0.15 wt/wt % and 1.0 wt/wt % of each of the non-identical peptides.

3. The microsphere formulation of claim 1, wherein each polymer microsphere comprises three non-identical peptides.

4. The microsphere formulation of claim 1, wherein each polymer microsphere further comprises an adjuvant.

5. The microsphere formulation of claim 4, wherein each polymer microsphere has a drug load of at least about 0.01 wt/wt % of the adjuvant.

6. The microsphere formulation of claim 1, wherein the biodegradable polymer has an inherent viscosity of between about 0.12 to about 3.0 dL/g.

7. The microsphere formulation of claim 1, wherein the biodegradable polymer is selected from the group consisting of a poly(lactide) polymer, a poly(lactide-co-glycolide) polymer, and a mixture thereof.

8. The microsphere formulation of claim 1, wherein the at least two non-identical peptides comprise SEQ ID NO: 1, SEQ ID NO: 2, and SEQ ID NO: 3.

9. The microsphere formulation of claim 4, wherein the adjuvant composition comprises a toll-like receptor 9 agonist adjuvant.

10. The microsphere formulation of claim 4, wherein the adjuvant composition comprises CpG.

11. The microsphere formulation of claim 1, further comprising a sugar.

12. The microsphere formulation of claim 11, wherein the sugar comprises D-mannitol.

13. The microsphere formulation of claim 11, wherein the sugar comprises D-(+)-mannose.

14. The microsphere formulation of claim 11, wherein the sugar is selected from the group consisting of D-(+)-mannose, D-mannitol, or a combination thereof, and wherein each polymer microsphere has a drug load of at least about 0.01 wt/wt % of the sugar.

15. The microsphere formulation of claim 1, wherein the polymer microspheres are characterized by a residual solvents content of less than about 1%.

16. A microsphere formulation, comprising:
polymer microspheres, each polymer microsphere comprising:
a. non-identical peptides, the non-identical peptides comprising SEQ ID NO: 1, SEQ ID NO: 2, and SEQ ID NO: 3; and
b. an adjuvant, the adjuvant comprising CpG; and
c. a biodegradable polymer selected from the group consisting of a poly(lactide) polymer, a poly(lactide-co-glycolide polymer, and a mixture thereof,
wherein each polymer microsphere has a drug load of at least about: (i) 0.1 wt/wt % of each of the peptides; and (ii) 0.01 wt/wt % of the adjuvant,
wherein the polymer microspheres have an average particle size (D₅₀) of less than about 12.6 μm, and
wherein the polymer microspheres are characterized by a residual solvents content of less than about 1%.

17. The microsphere formulation of claim 16, further comprising a sugar, wherein the sugar is selected from the group consisting of D-(+)-mannose, D-mannitol, or a combination thereof, and wherein each polymer microsphere has a drug load of at least about 0.01 wt/wt % of the sugar.

18. The microsphere formulation of any of claim 16, wherein each polymer microsphere has a drug load of at least about: (i) 0.5 wt/wt % of each of the peptides; and (ii) 0.02 wt/wt % of the adjuvant.

19. A method for making a microsphere formulation comprising at least two non-identical peptides, the method comprising:
(A) contacting, in an organic solvent:
(i) at least two non-identical peptides;
(ii) an adjuvant; and
(iii) a biodegradable polymer, to form a dispersed phase;
(B) combining the dispersed phase with a continuous phase comprising water and surfactant in a homogenizer to form an emulsion;
(C) removing the organic solvent from the emulsion to form a microsphere formulation essentially free of organic solvent; and
(D) subjecting the substantially organic solvent-free microsphere formulation to freeze-drying.

20. The method of claim 19, wherein the surfactant comprises polyvinyl alcohol.

21. The method of claim 20, wherein the polyvinyl alcohol is present in a concentration of 0.35% by weight in water.

22. The method of claim 19, wherein the at least two non-identical peptides are selected from the group consisting of SEQ ID NO: 1, SEQ ID NO: 2, and SEQ ID NO: 3, and combinations thereof.

23. The method of claim 19, wherein the adjuvant composition comprises a toll-like receptor 9 agonist adjuvant.

24. The method of claim 19, wherein each non-identical peptide is present in the each microsphere in an amount of about 0.5% to about 1.0% by weight of the microspheres, not including minimal residual solvent.


SEQUENCE LISTING (3 sequences)

SEQ ID NO: 1 - protein, 9 aa, SARS-CoV-2
LSPRWYFYY

SEQ ID NO: 2 - protein, 9 aa, SARS-CoV-2
LLLDRLNQL

SEQ ID NO: 3 - protein, 9 aa, SARS-CoV-2
KTFPPTEPK